(19) United States
(12) Patent Application Publication
(10) Pub. No.: US 2024/0260974 A1
(43) Pub. Date: Aug. 8, 2024

(54) Acetabular Augment Reaming Guide

(71) Applicant: Howmedica Osteonics Corp., Mahwah, NJ (US)

(72) Inventors: Michael Shabelman, Fair Lawn, NJ (US); Caroline Atkinson, Hackensack, NJ (US)

(21) Appl. No.: 18/429,678

(22) Filed: Feb. 1, 2024

Related U.S. Application Data
(60) Provisional application No. 63/483,087, filed on Feb. 3, 2023.

(51) Int. Cl. A61B 17/16

(52) U.S. Cl. CPC A61B 17/1666; A61B 17/1631

(57) ABSTRACT

An acetabular wedge augment reaming tool includes a scoop assembly, an acetabular reamer assembly and a tool guide. The scoop assembly has a scoop configured to be received within an acetabulum and a scoop shaft extending from the scoop. The acetabular reamer assembly has a reamer head and a reamer shaft extending from the reamer head. The tool guide includes a reamer guide housing and a reamer guide positioned within the reamer guide housing and moveable along a first axis relative to the reamer guide housing. The reamer shaft of the acetabular reamer assembly is slidably connected to the reamer guide such that it is moveable along a second axis relative to the reamer guide and the scoop shaft of the scoop assembly is connected to the reamer guide housing.

### CROSS-REFERENCE TO RELATED APPLICATIONS

This application claims the benefit of the filing date of U.S. Provisional Patent Application No. 63/483,087 filed Feb. 3, 2023, the entire disclosure of which is hereby incorporated herein by reference.

### BACKGROUND OF THE INVENTION

Joint replacement surgery is a common orthopedic procedure for joints such as the shoulder, hip, knee, ankle and wrist. Prior to implanting prosthetic components in a joint of a patient, a surgeon generally has to resect at least a portion of the patient's native bone in order to create a surface and/or recess or void for supporting, accepting, or receiving at least a portion of the prosthetic components being implanted. Generally, a surgeon only resects the amount of bone that is needed in order to properly implant the prosthetic components in the joint because once native bone is resected from a joint, it is gone forever. Thus, the surgeon typically attempts to maintain as much of the native structural integrity of the joint as he or she can during the resection process.

However, issues of additional loss of native bone near the joint being replaced are often encountered in revision procedures or in circumstances of trauma or significant disease. With respect to revision procedures, this bone loss is typically due to movement of the primary prosthesis after its initial implantation or even degeneration or further degeneration of the bone, which can form bone defects or bone voids that have unpredictable and non-uniform shapes. In addition, revision procedures often involve the removal of additional bone, which makes maintaining or otherwise restoring the structural integrity often afforded by native bone of great importance.

Thus, when bone defects are observed, it is standard surgical practice to fill or augment those defects as part of the surgical procedure in order to help support the joint prosthesis. The preferred practice is to fill those defects with weight bearing augments or void fillers, typically made of an implant-grade metal such as titanium. However, because the bone defects are typically irregular in shape, some preparation of the defect area is typically required prior to implantation of the augment. However, such preparation is often performed unguided using common surgical instruments, such as ronguers, which are not particularly adapted for contouring the bone to match the augment, thus, requiring a time-consuming iterative approach of cutting, evaluating, cutting again and so forth until a reasonable match is achieved. The difficulty of current approaches to bone preparation is further compounded by the fact that augments and the joint prostheses they support typically come in a variety of sizes and are typically adapted to be complimentary to each other. Poor bone preparation can disrupt this complimentary relationship. Thus, further improvements are desirable.

### BRIEF SUMMARY

In a first aspect, the present disclosure relates to an acetabular wedge augment reaming tool. In a first example of a first embodiment of the first aspect, the acetabular wedge augment reaming tool includes a scoop assembly, an acetabular reamer assembly, and a tool guide. The scoop assembly has a scoop configured to be received within an acetabulum and a scoop shaft extending from the scoop. The acetabular reamer assembly has a reamer head and a reamer shaft extending from the reamer head. The tool guide comprises a reamer guide housing and a reamer guide positioned within the reamer guide housing and moveable along a first axis relative to the reamer guide housing. The reamer shaft of the acetabular reamer assembly is slidably connected to the reamer guide such that it is moveable along a second axis relative to the reamer guide and the scoop shaft of the scoop assembly is connected to the reamer guide housing. Moving the reamer guide along the first axis moves the reamer assembly closer to or further away from the scoop assembly.

In a second example, the first example of the first embodiment may be further defined wherein the scoop is modularly connected to a distal end of the scoop shaft of the scoop assembly. In a third example, the first example of the first embodiment may be further defined wherein the scoop further includes a semi-spherical exterior that defines an outer diameter. In a fourth example, the third example of the first embodiment may by further defined wherein the scoop further includes a semi-spherical cavity that defines an inner diameter. In a fifth example, the first example of the first embodiment may be further defined wherein the scoop includes a rim extending outwardly from a perimeter of the scoop that is interrupted by a cutout. In a sixth example, the fifth example of the first embodiment may be further defined wherein the scoop shaft is modularly connected to a first side of the scoop and the cutout is positioned at a second side of the scoop opposite the first side.

In a seventh example, the second example of the first embodiment may be further defined wherein the scoop assembly includes a housing shaft connected to and extending from the reamer guide housing, the scoop shaft being received within the housing shaft so that the distal end of the scoop shaft extends from a distal end of the housing shaft. In an eight example, the seventh example of the first embodiment may be further defined wherein a scoop knob is disposed at a proximal end of the scoop shaft, and the distal end of the scoop shaft is threaded for threaded connection with the scoop.

In a ninth example, the first example of the first embodiment may further comprise a handle assembly having a handle and a handle shaft connected to the reamer guide housing. In a tenth example, the ninth example of the first embodiment may be further defined wherein handle assembly extends proximally from the reamer guide housing at an angled offset.

In an eleventh example, the first example of the first embodiment may be further defined wherein the acetabular reamer shaft includes a mechanical stop positioned along a length thereof and configured to limit sliding movement thereof relative to the reamer guide. In a twelfth example, the first example of the first embodiment may be further defined wherein the reamer housing includes housing rails at least partially defining a cavity within the reamer housing, the reamer guide being positioned within the cavity and engaging the housing rails from within the cavity of the reamer housing. In a thirteenth example, the twelfth example of the first embodiment may further comprise an adjustment knob and a threaded shaft extending from the adjustment knob into the cavity of the housing and engaging the reamer guide such that rotation of the adjustment knob slides the reamer guide along the housing rails and along the first axis.

In a fourteenth example, the first example of the first embodiment may be further defined wherein the scoop has a convex exterior having a first diameter corresponding to a reamed diameter of an acetabulum. In a fifteenth example, the fourteenth example of the first embodiment may be further defined wherein the reamer head has a second diameter. In a sixteenth example, the fifteenth example of the first embodiment may be further defined wherein the reamer head has a second diameter.

In a seventeenth example, the first example of the first embodiment may be further defined wherein the tool guide comprises markings corresponding to an acetabular reamer position along the first axis. In a eighteenth example, the seventeenth example of the first embodiment may be further defined wherein the markings includes a first acetabular cup size marking are disposed on a proximal side of the reamer guide and plurality of first wedge width markings and a plurality of second wedge width markings disposed on the reamer guide housing, the first acetabular cup size marking corresponding to a first range of acetabular cup sizes having a first and second cup size, the first wedge width markings each corresponding to a first acetabular augment wedge width and separately to the first and second cup sizes of the first range of acetabular cup sizes, and the second wedge width markings each corresponding to a second acetabular augment wedge width and separately to the first and second cup sizes of the first range of acetabular cup sizes. In a nineteenth example, the eighteenth example of the first embodiment may be further defined wherein the first range of acetabular cup sizes is 45-48, 49-52, 53-56, 57-60, 61-64, or 65-68 millimeters. In a twentieth example, the eighteenth example of the first embodiment may be further defined wherein the first and second wedge width markings each correspond to a wedge augment diameter of 46, 50, 54, 58, 62, or 66 millimeters if the range of acetabular cup sizes is respectively 45-48, 49-52, 53-56, 57-60, 61-64, or 65-68 millimeters. In a twenty-first example, the eighteenth example of the first embodiment may be further defined wherein the first wedge width and second wedge width are separately any one of 15, 20 and 25 millimeters.

In a twenty-second example, the first example of the first embodiment may be further defined wherein a reamer guide knob is configured on a first side of the reamer guide housing and locks the reamer guide in a desired reaming position. In a twenty-third example, the first example of the first embodiment may be further defined wherein a locking pin is positioned on a second side of the reamer guide housing and is moveable from a first position in which the reamer guide is moveable relative to the reamer guide housing and a second position in which the locking pin locks the reamer guide in a desired reaming position. In a twenty-fourth example, the first example of the first embodiment may be further defined wherein the reamer head is spherical. In a twenty-fifth example, the first example of the first embodiment may be further defined wherein the reamer head is planar.

In a twenty-sixth example, the first example of the first embodiment may further comprise a shim positioned within a cavity of the reamer guide housing, the shim being configured to limit movement of the reamer guide along the first axis. In a twenty-seventh example, the twenty-sixth example of the first embodiment may be further defined wherein the shim is sized such that when the shim is positioned within the cavity of the reamer guide housing to define a spacing between the scoop assembly and the acetabular reamer assembly, operation of the scoop assembly and the acetabular reamer assembly on a bone surface produces surfaces sized to receive an acetabular wedge augment having a first predetermined size and an acetabular cup having a second predetermined size.

In a first example of a second embodiment, a reaming tool comprises a guide assembly, a reaming assembly, and an indexing assembly. The guide assembly has a housing and a reamer guide, the housing having a first end, a second end, and a channel located between the first and second ends, the reamer guide being disposed within the channel and moveable therein along a first axis. The reaming assembly has a reamer head and a reamer shaft extending from the reamer head, an elongate dimension of the reamer shaft extending along a second axis and being coupled to the reamer guide. The indexing assembly has an indexing head configured to be received within a bone void and an indexing shaft extending from the indexing head, an elongate dimension of the indexing shaft extending along a third axis and being coupled to the reamer guide housing. Moving the reamer guide along the first axis from a first position to a second position moves the reamer assembly relative to the indexing assembly, and wherein the second and third axes are parallel in the first and second positions. In a second example, the first example of the second embodiment may be further defined wherein the first axis is perpendicular to the second and third axes.

In a first example of a third embodiment, a kit for an acetabular wedge augment reaming tool comprises a scoop assembly, an acetabular reamer assembly, a tool guide, and a plurality of shims. The scoop assembly includes a scoop configured to be received within an acetabulum, the scoop having a diameter corresponding to a diameter of an acetabular cup implant. The acetabular reamer assembly has a reamer head, the reamer head having a diameter corresponding to a diameter of an acetabular augment. The tool guide comprises a reamer guide housing and a reamer guide configured to be positioned within the reamer guide housing. Each shim of the plurality of shims is configured to be positioned within a cavity of the reamer guide housing, and each shim is configured to limit movement of the reamer guide within the reamer guide housing. The scoop assembly and the acetabular reamer assembly are adapted for operative connection to the tool guide along with a first shim of the plurality of shims such that the first shim is disposed between the scoop assembly and the acetabular reamer assembly. In a second example, the first example of the third embodiment may further be defined wherein the reamer guide is slidable within the reamer guide housing such that a second shim of the plurality of shims having a different size than the first shim is disposable between the scoop assembly and the acetabular reamer assembly.

In a second aspect, the present disclosure relates to a method of preparing an acetabular defect for receipt of an augment implant. In a first example of a first method of the second aspect, the method includes reaming an acetabulum to a first diameter; inserting a scoop of a scoop assembly into the reamed acetabulum, the scoop assembly having a scoop and a scoop shaft, the scoop having a second diameter equal to the first diameter of the reamed bone; adjusting a reamer guide of a guide assembly and a reamer assembly connected to the reamer guide along a first axis from a first position to a second position relative to the scoop assembly, the reamer assembly having a reamer shaft and a reamer head, the reamer shaft being parallel to the scoop shaft in the first and second positions; driving the reamer head into a bone defect adjacent the acetabulum; implanting an acetabular implant into acetabulum; and implanting the augment implant in the bone defect.

In a second example, the first example of the first method further comprises trialing the bone defect to determine an augment size. In a third example, the first example of the first method may be further defined wherein, in the adjusting step, the reamer guide is moved until markings of the reamer guide corresponding to the augment size are aligned. In a fourth example, the third example of the first method may be further defined wherein the adjusting step includes turning a knob which drives the reamer guide along the first axis relative to a reamer guide housing of the guide assembly, the shaft of the scoop being coupled to the reamer guide housing. In a fifth example, the fourth example of the first method may be further defined wherein the reamer shaft defines a second axis perpendicular to the first axis.

### BRIEF DESCRIPTION OF THE DRAWINGS

The features, aspects, and advantages of the present invention will become better understood with regard to the following description, appended claims, and accompanying drawings in which:

FIG. 1 is a perspective view of an acetabular wedge augment reaming tool according to an embodiment of the present disclosure.

FIG. 2A is a first side view of the acetabular wedge augment reaming tool of FIG. 1.

FIG. 2B is a second side view of the acetabular wedge augment reaming tool of FIG. 1.

FIG. 3A is a front view of the acetabular wedge augment reaming tool of FIG. 1.

FIG. 3B is a rear view of the acetabular wedge augment reaming tool of FIG. 1.

FIGS. 3C-3D are cross-sectional views of the tool guide taken along line 3C of FIG. 3B.

FIGS. 3E-3F are cross-sectional views of the scoop taken along line 3E of FIG. 3B.

FIG. 4A is a bottom view of the acetabular wedge augment reaming tool of FIG. 1.

FIG. 4B is a top view of the acetabular wedge augment reaming tool of FIG. 1.

FIG. 5A. is a perspective view of a scoop assembly of the acetabular wedge augment reaming tool of FIG. 1.

FIG. 5B is a perspective view of an inner scoop shaft and scoop of the scoop assembly of FIG. 5A.

FIG. 6A is a perspective view of a scoop of the acetabular wedge augment reaming tool of FIG. 1.

FIG. 6B is a top view of the scoop of FIG. 6A.

FIG. 6C is a front view of the scoop of FIG. 6A.

FIG. 7A is a perspective view of a handle connected to the scoop assembly of the acetabular wedge augment reaming tool of FIG. 1.

FIG. 7B is a perspective view of the handle of the acetabular wedge augment reaming tool of FIG. 1.

FIG. 8A is a perspective view of an acetabular reamer assembly of the acetabular wedge augment reaming tool of FIG. 1.

FIG. 8B is a perspective view of the acetabular reamer shaft of the acetabular reamer assembly of FIG. 8A.

FIG. 9A is a proximal view of a tool guide and markings of the acetabular wedge augment reaming tool of FIG. 1.

FIG. 9B is an enlarged view of the markings of FIG. 9A.

FIG. 10 is a perspective view of a wedge augment according to an embodiment of the present disclosure.

FIG. 11 illustrates reaming an acetabulum.

FIGS. 12A and 12B illustrate trialing the reamed acetabulum of FIG. 11 and an adjacent defect thereof.

FIG. 13 illustrates assembling a scoop to the acetabular wedge augment reaming tool of FIG. 1.

FIG. 14 illustrates adjusting the acetabular wedge augment reaming tool of FIG. 1.

FIGS. 15A-15B illustrate reaming the defect of FIG. 12 with the acetabular reaming tool of FIG. 1.

FIG. 16A depicts a reamed acetabulum and adjacent defect.

FIG. 16B illustrates implanting an acetabular implant and wedge implant within the reamed acetabulum and adjacent defect of FIG. 16A.

FIG. 17 is a perspective view of an acetabular wedge augment reaming tool according to another embodiment of the present disclosure.

FIG. 18A is a perspective view of a planar reamer of the acetabular wedge augment reaming tool of FIG. 17.

FIG. 18B is a rear view of the planar reamer of FIG. 18A.

FIGS. 19A-19C illustrate a method of using the acetabular wedge augment reaming tool of FIG. 18.

FIG. 20A is a perspective view of an alternate embodiment of a scoop of the acetabular wedge augment reaming tool of FIG. 1.

FIG. 20B is a top view of the scoop of FIG. 20A.

FIG. 20C is a front view of the scoop of FIG. 20A.

FIG. 21 is a perspective view of an alternate embodiment of an outer scoop shaft of the acetabular wedge reaming tool of FIG. 1.

FIG. 22 is a perspective view of an acetabular wedge augment reaming tool according to another embodiment of the present disclosure.

FIG. 23 is a side view of the acetabular wedge augment reaming tool of FIG. 22.

FIG. 24A is a perspective view of a shim of the acetabular wedge augment reaming tool of FIG. 22.

FIG. 24B is a front view of the shim of FIG. 24A.

FIG. 24C is a top view of the shim of FIG. 24A.

FIG. 24D is a bottom view of the shim of FIG. 24A.

FIG. 25A is a side view of an acetabular reamer shaft of the acetabular wedge augment reaming tool of FIG. 22.

FIG. 25B is a side view of the acetabular reamer shaft of FIG. 25A with a mechanical stop hidden.

FIG. 25C is a cross sectional view of a mechanical stop of the acetabular reamer shaft of FIG. 25A.

### DETAILED DESCRIPTION

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended. As used herein, the terms “substantially,” “generally,” “approximately,” and “about” are intended to mean that slight deviations from absolute are included within the scope of the term so modified, such as deviations of up to 10% greater or lesser than absolute. All vertical directional terms, such as “up,” “down,” “above,” “below,” “vertical,” or “height” used in the following description refer only to the orientation of features as depicted in the figure being described. Such directional terms are not intended to suggest that any features of the devices described herein must exist in any particular orientation when constructed.

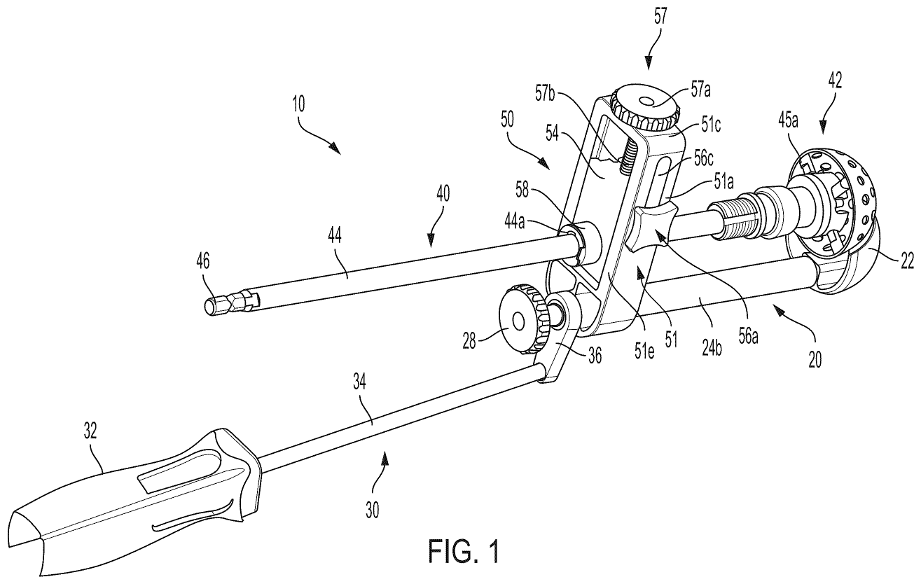

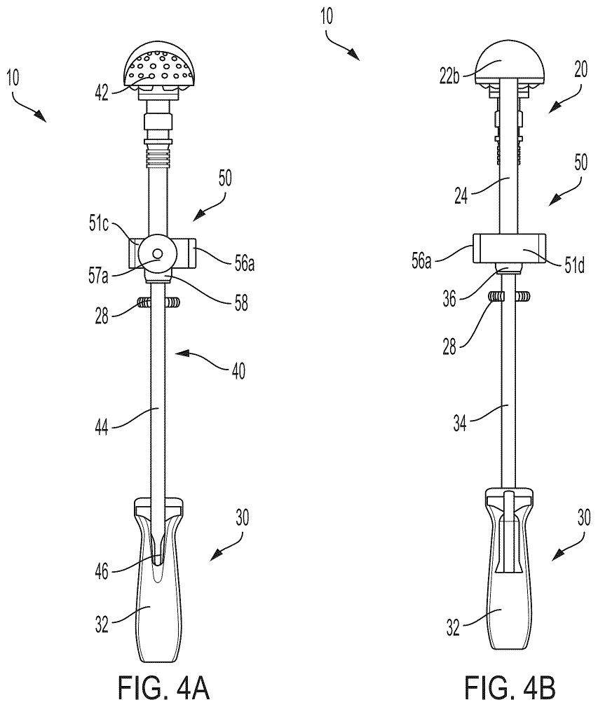

FIGS. 1A-9B depict an acetabular wedge augment reaming tool 10 according to one embodiment of the present disclosure. Acetabular wedge augment reaming system is particularly configured for addressing cavitary defects of an acetabulum. Acetabular wedge augment reaming tool 10 includes a scoop or index assembly 20, a handle assembly 30, an acetabular reamer assembly 40, and a tool guide 50.

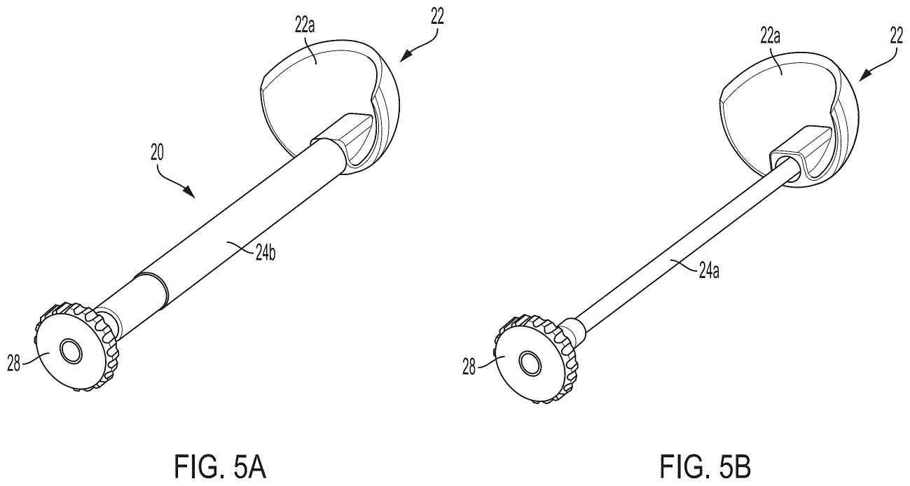

Scoop assembly or index assembly 20 generally includes an inner shaft 24a, an outer shaft 24b, and a scoop 22, as best shown in FIGS. 5A and 5B.

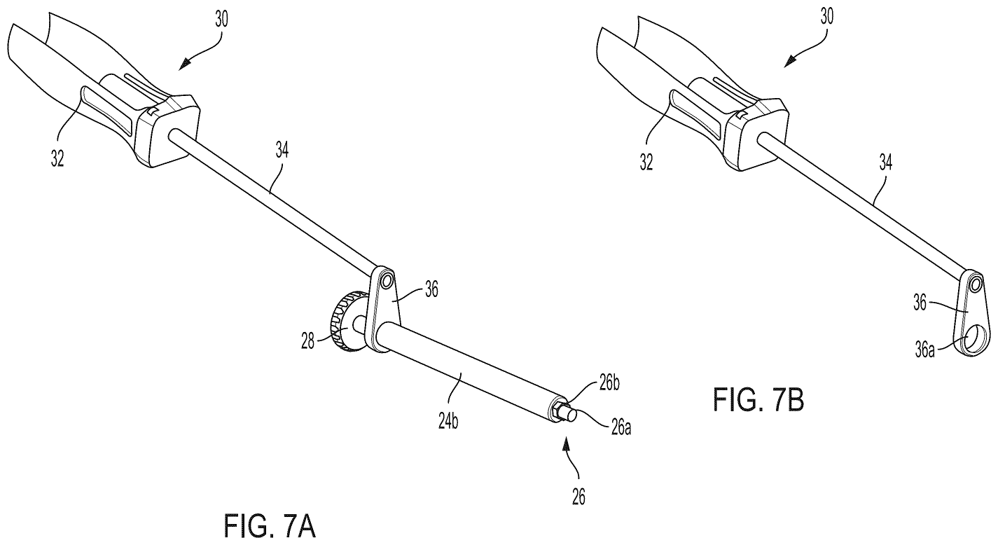

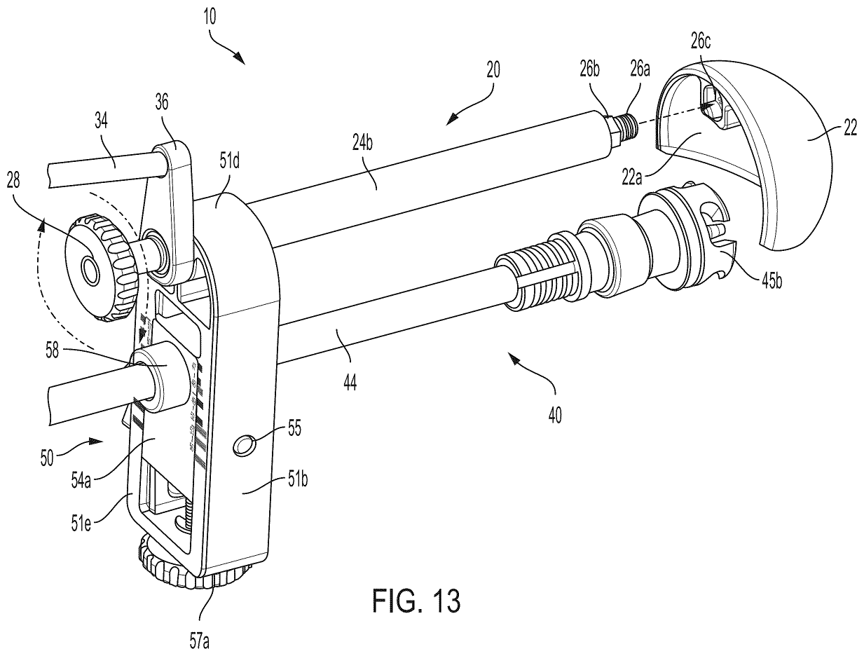

Inner shaft or scoop shaft 24a includes a first end and a second end. The first end or distal end includes a threaded tip 26a that is configured to threadedly connect to scoop 22, as best shown in FIGS. 7A and 13. The second end or proximal end includes a knob 28 that allows an operator to manually rotate inner shaft 24a to facilitate threaded connection with scoop 22, as shown in FIG. 5B.

Outer shaft or scoop housing 24b is a cannulated shaft. In this regard, outer shaft 24b has an opening extending through its entire length which is configured to rotatably receive inner shaft 24a. A first end or distal end of outer shaft 24b includes an anti-rotation feature 26b, which in the particular embodiment depicted is a hex-shaped tip, as best shown in FIGS. 7A and 13. Such anti-rotation feature 26b is configured to be received within a corresponding opening in scoop 22 and prevent rotation of scoop 22 relative to outer shaft 24b, as shown in FIG. 5A. The length of outer shaft 24b is such that when inner shaft 24a is fully received therein, threaded tip 26a of inner shaft 24a extends from the distal end of outer shaft 24b, as shown in FIG. 7A. In this configuration, anti-rotation feature 26b of outer shaft 26b and threaded tip 26a of inner shaft 24a together form a scoop modular connection 26.

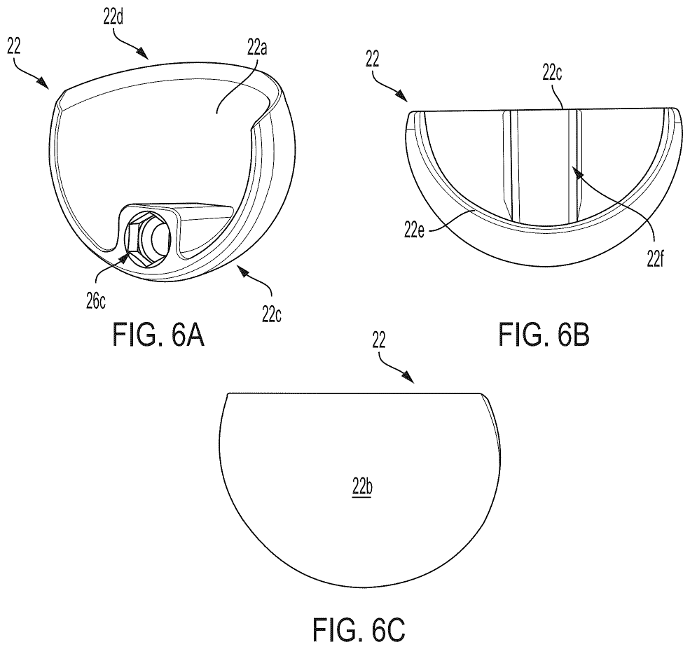

Scoop or indexer head 22 is defined by an inner surface 22a, an outer surface 22b, first side 22c, a second side 22d, a cutout 22e and a modular connection or first mating feature 26c, as best shown in FIGS. 6A-6C. The outer surface 22b generally defines a semi-spherical exterior that defines an outer diameter. Similarly, the inner surface 22a generally defines a semi-spherical interior that defines an inner diameter. The modular connection 26c is generally positioned proximally to the first side 22c of the scoop 22. The modular connection 26c is configured to mate with modular connection 26. In this regard, modular connection 26c of scoop 22 is in the form of a hex-shaped opening with a threaded interior. As such, modular connection 26c of scoop 22 is configured to engage both threaded tip 26a of inner shaft 24a and anti-rotation feature 26b of outer shaft 24b. The second side 22d of the scoop 22 is located opposite the first side 22d and is generally defined by a rim 22e that is interrupted by a cutout 22f. Such cutout 22f creates a clearance space for a reamer head, as described further below.

Referring to FIGS. 5A and 5B, assembly 20 is assembled by engaging anti-rotation feature 26b of outer shaft 24b with scoop modular connection 26c. Inner shaft 24a is inserted through outer shaft 24b so that threaded tip 26a engages scoop modular connection 26c. Rotation of inner shaft 24a via knob 28 threadedly secures inner shaft 24a to scoop 22. Further, rotation of the knob 28 causes a compression of outer shaft 24b between scoop 22 and knob 28. In other words, when threaded end 26a of inner shaft 24a is advanced into scoop 22, scoop 22 is drawn tighter onto outer scoop shaft 24b and anti-rotation feature 26b thereof which helps to further secure scoop to the shafts 24a-b and provides stability to scoop 22 so that scoop 22 does not wobble during reaming operations. Anti-rotation features 26a of outer shaft 24b help prevent rotation of scoop 22 as inner shaft 24a is connected to scoop 22. Thus, as assembled, scoop 22 is engaged to scoop inner shaft 24a and outer shaft 24b concurrently. FIG. 5A shows scoop inner shaft 24a within the scoop housing 24a such that knob is disposed at an opposite end of scoop housing 24a than scoop 22. FIG. 5B shows scoop 22 connected to scoop inner shaft 24a by scoop modular connection 26c and a scoop shaft modular connection 26a.

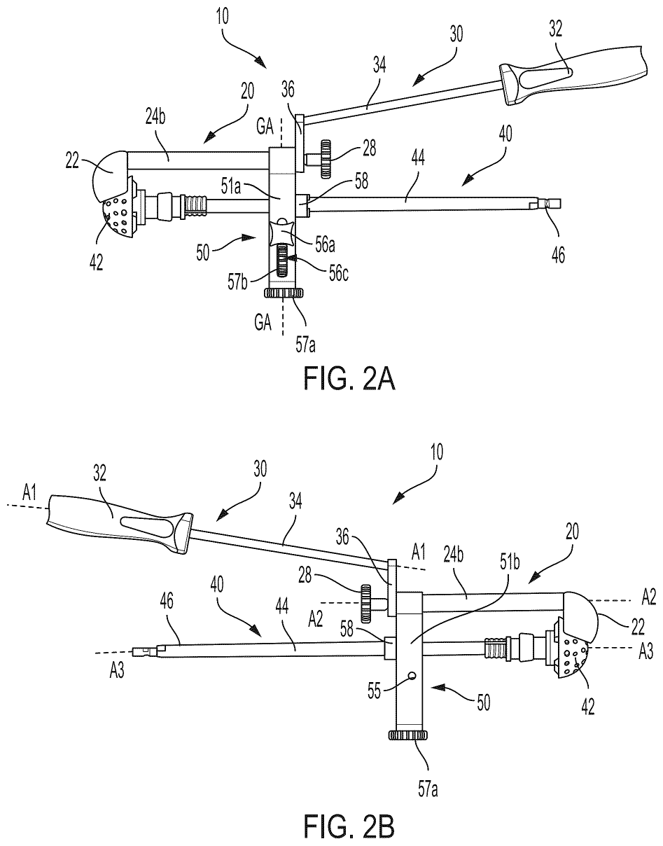

Handle assembly 30 generally includes a handle 32, a handle shaft 34, and a handle connector 36, as best shown in FIGS. 7A and 7B. Handle connector 36 is a plate-like structure that has a first end connected to a distal end of handle shaft 34 and a second end connected to outer scoop shaft 24b. In this regard, handle connector 36 has an opening 36a (see FIG. 7B) that receives outer shaft 24b, such as in press-fit manner, to securely connect handle connector 36 to outer scoop shaft 24b. Additionally, in the embodiment depicted, handle shaft 34 extends proximally from the handle connector 36 at an angle or slant that is not parallel with scoop shaft 24b. In other words, handle shaft 34 is arranged such that a longitudinal axis A1 thereof is oriented at an oblique angle relative to a longitudinal axis A2 of outer scoop shaft 24b, as best shown in FIG. 2B. In this regard, outer shaft 24b is generally perpendicular to a longitudinal axis of handle connector 36 while handle shaft 34 is obliquely oriented with respect to handle connector 36. Handle 32 is connected to a proximal end of handle shaft 34 and is configured to be a held by a user in operation of the procedure using the acetabular wedge augment tool 10.

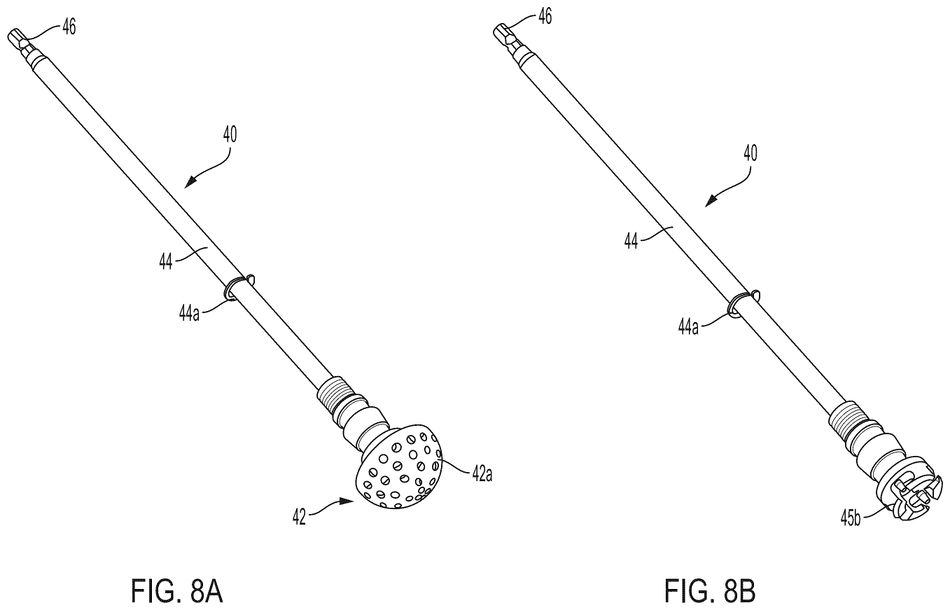

Acetabular reamer assembly 40 generally includes an acetabular reamer head 42 and acetabular reamer shaft 44, as shown in FIGS. 8A and 8B. The reamer head or cutting head 42 has a convex exterior with cutting features 142b and a concave interior with a mating feature 45a disposed therein. The mating feature 45a, as shown in FIG. 1, is in the form of elongate struts extending across the concave interior of reamer head 42. Acetabular reamer shaft 44 defines a reamer axis A3 and includes a corresponding mating feature 45b at its distal end which, in the embodiment depicted, comprises a plurality of tapered hooks which are configured to latch onto the elongate struts of mating feature 45a through rotational engagement. In this regard, reamer head 42 is connected to reamer shaft 44 by pushing reamer shaft down and applying a quarter-turn to lock mating features 45a-b together. The reamer head 42 is disconnected from the mating feature by rotating the reamer head a quarter-turn in a clockwise direction and pulling back on reamer shaft 44. Thus, mating features 45a-b together form a bayonet-style connection. However, in other embodiments of reamer assembly 40, mating features 45a-b may be in the form of a groove and lip for a snap-fit connection, corresponding threads for a threaded connection, or the like, for example. Reamer shaft 44 includes a tool connector 46 at its proximal end which may be connected for engagement with a hand-held power tool (not shown) that is able to rotate and drive the reamer assembly 40 into bone. The acetabular reamer assembly 40 further includes a reamer guide mechanical stop 58 connected to the acetabular reamer shaft 44 at a location along its length desirable to ensure an appropriate reaming depth. Thus, mechanical stop 58 prevents the unrestricted sliding movement of the acetabular reamer assembly 40 within the opening of the reamer guide 54 of the tool guide 50, as described further below.

Tool guide 50 generally includes a reamer guide 54, a reamer guide housing 51, a reamer adjustment assembly 57, and a reamer guide locking assembly 56. The housing 51 includes reamer guide rails or sidewalls 51a-b that at least partially define a cavity 52 and respective first and second sides of housing 51. First sidewall 51a defines a slot 56c extending therethrough for receipt of a shaft 56b of locking assembly 56. Reamer guide housing 51 also includes a first end wall 51c and a second end wall 51d that at least partially define cavity 52. First end wall 51c defines a first end of housing 51 and defines a first opening extending therethrough for receipt a shaft 57b of reamer adjustment assembly 57. Second end wall 51d defines a second opening that extends in a direction transverse to a longitudinal axis of reamer guide housing 51. The second opening is configured to receive outer scoop shaft 24b, such as in a press-fit manner.

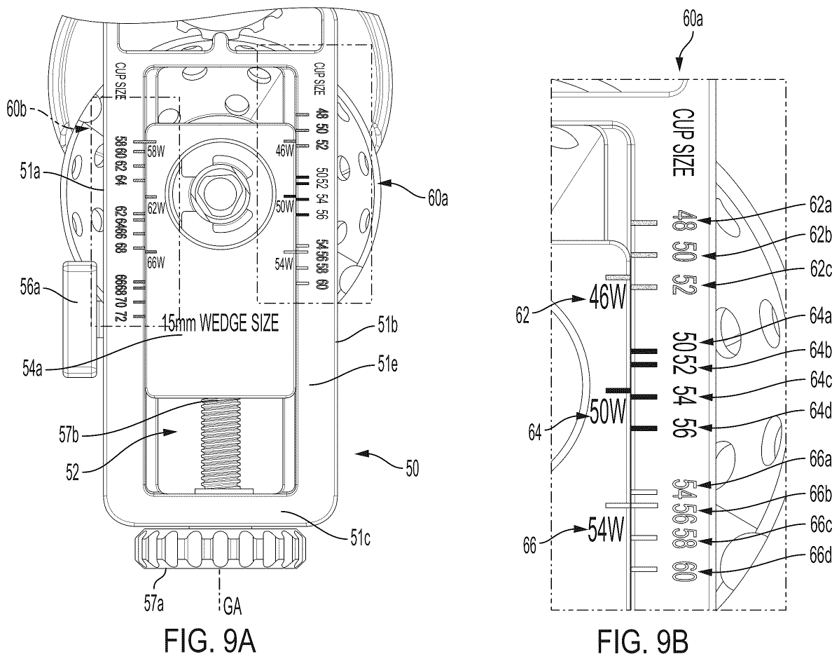

Reamer guide or shuttle 54 is positioned within cavity 52 of reamer guide housing 51 and engages sidewalls 51a-b for sliding axial movement, such as in a tongue and groove arrangement, for example. In this regard, guide 54 is moveable along a guide axis GA of housing 51, as best shown in FIG. 2A. Reamer guide 54 is secured to housing 51 and within cavity 42 via a locking pin 55 which prevents reamer guide 54 from falling out of cavity 42 while allowing for translation of reamer guide 54 within cavity 42. Reamer guide 54 also includes a first opening extending therein and along the guide axis GA, a second opening extending in a direction transverse to the first opening and guide axis GA, and a third opening extending transverse to the first and second openings. The first opening (not shown) is threaded and configured to threadedly receive a threaded shaft 57b of reamer adjustment assembly, as best shown in FIGS. 1 and 9A. The second opening (not shown) is a smooth bore that is configured to slidingly receive reamer shaft 44 of reamer assembly 40 such that the second opening is coaxial with reamer axis A3 when reamer shaft 44 is received therein, as best shown in FIGS. 1 and 2B, and the third opening is a threaded opening that receives a threaded shaft 56b of guide locking assembly 56.

Reamer adjustment assembly 57 includes an adjustment knob 57a and an adjustment shaft 57b. Adjustment shaft or threaded shaft 57b extends from the adjustment knob 57a into the cavity 52 of the housing 51 to engage the corresponding first opening (i.e., threaded opening) in reamer guide 54. Rotation of the adjustment knob 57a translates reamer guide 54 along the guide rails 51a-b and along Guide Axis GA.

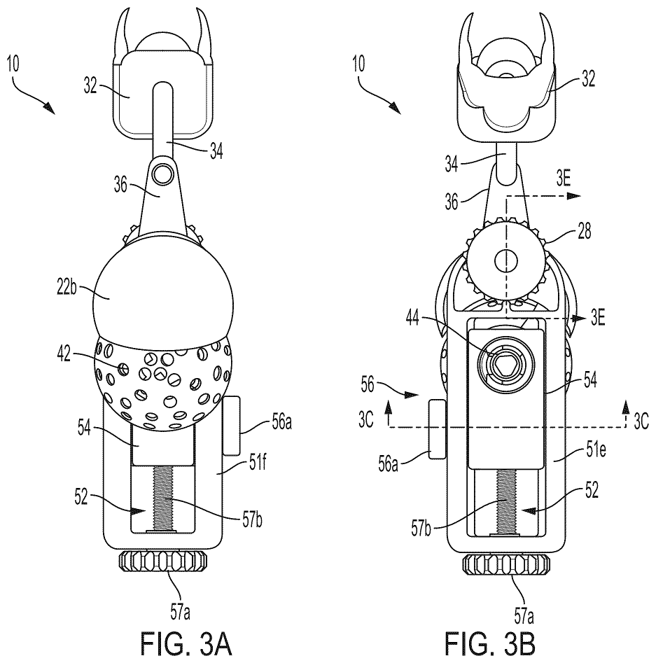

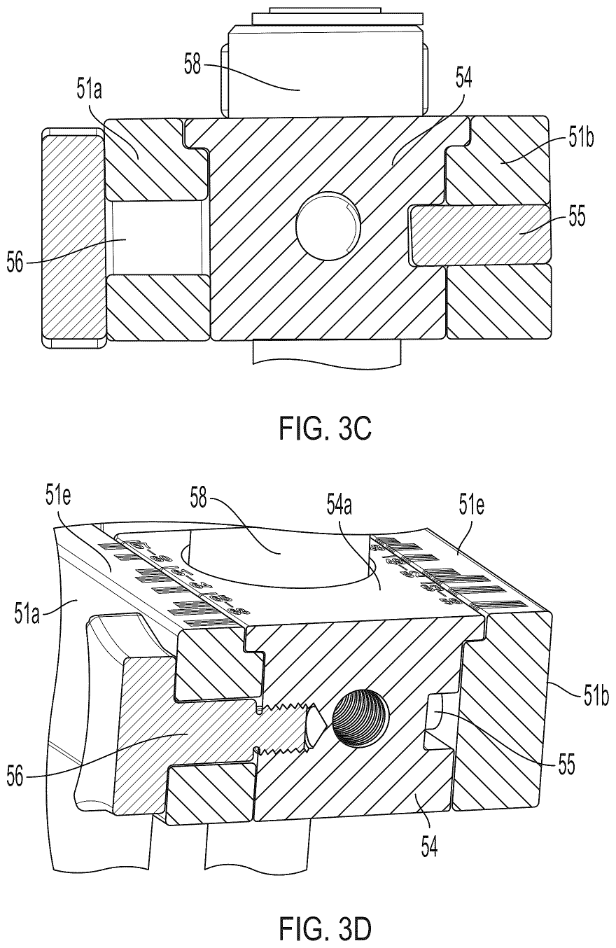

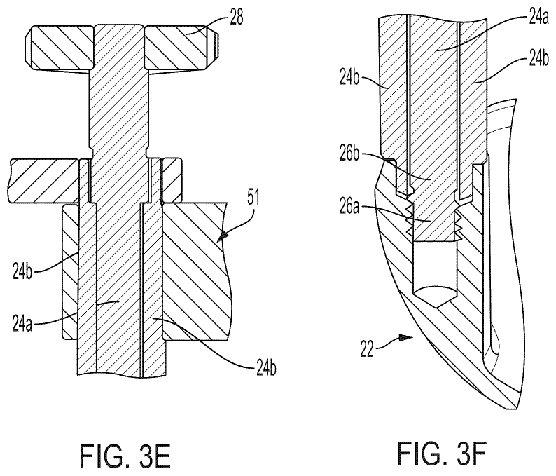

Reamer guide locking assembly 56 generally includes a locking knob 56a and a threaded shaft 56b, as best shown in FIGS. 3C-3D. Locking shaft or threaded shaft 56b is connected to locking knob 56a and extends through groove 56c in housing 51 and into the third opening in reamer guide 54. Turning locking knob 56a in a first direction secures reamer guide 54 to first sidewall 51a of housing 51 to rigidly lock reamer guide 54 from movement, while turning locking knob 56a in an opposite second direction unlocks guide 54 so that further adjustments can be made.

When acetabular wedge augment reaming tool 10 is assembled, scoop outer shaft 24b is connected to the second opening within second end wall 51d of tool housing 51, and inner scoop shaft 24a extends through outer scoop shaft 24b. Scoop 22 is connected to the respective distal ends of inner and outer scoop shafts 24a-b.

The handle 32 of handle assembly 30 is connected to the handle shaft 34 at a proximal end of the handle assembly 30 and a proximal end of the acetabular wedge augment reaming tool 10. The handle shaft 34 is connected to a distal end of the handle 32 and extends distally towards the tool guide 50. The first end of the handle connector 36 is connected to a distal end of the handle shaft 34, and a second end of the handle connector 36 is connected to a proximal end of the scoop outer shaft 24b.

The reamer head 42 of reamer assembly 40 is connected to the reamer shaft 44 at a distal end thereof. The reamer shaft 44 of reamer assembly passes through the second opening (not shown) of a reamer guide 54 so that the reamer shaft axis A3 is parallel with the longitudinal axis of scoop assembly axis A2, as best shown in FIG. 2B. Reamer assembly 40 is moveable along a guide axis GA between a plurality of positions, such as a first and second position, via operation of reamer adjustment assembly 57. It is noted that throughout this translational movement of reamer assembly 40 along guide axis GA, reamer axis A3 and scoop assembly axis A2 remain parallel. Mechanical stop 58 of reamer assembly 40 is positioned proximal of reamer guide 54 while reamer head 42 is positioned distal of reamer guide 54. Thus, mechanical stop 58 acts as a depth limiter when it abuts a proximal surface of reamer guide 54.

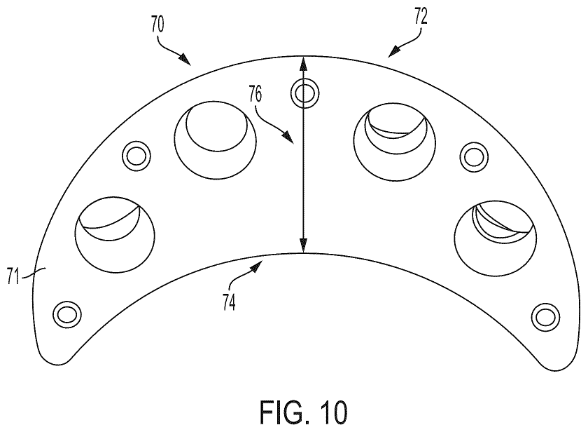

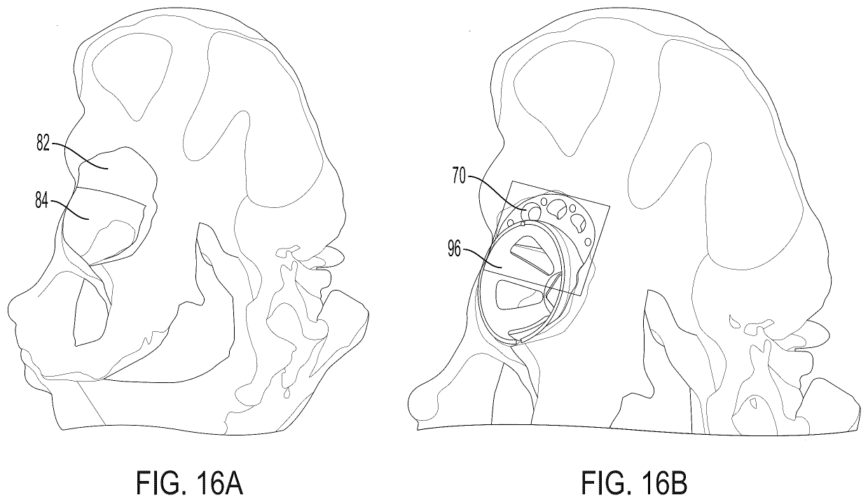

Scoop assembly 20 acts as an indexer in that scoop 22 engages an acetabulum and sets an origin from which reamer assembly 40 refers in order to appropriately ream a void for an acetabular augment. An exemplary acetabular augment 70 is shown in FIG. 10 and includes a concave inner surface 74 and a convex outer surface 72. Concave inner surface 74 defines an inner diameter of augment 70, convex outer surface 72 defines an outer diameter of augment 70, and an augment thickness 76 is defined between the convex and concave surfaces 72, 74. The inner diameter of augment 70 is generally configured to correspond with an outer diameter of an acetabular cup prosthesis 96, an example of which is depicted in FIG. 16B. However, the inner diameter of augment 70 need not precisely match the outer diameter of the acetabular prosthesis 96 as the augment 70 may be cemented to the prosthesis 96. Thus, the cement may account for discrepancies in diameters between acetabular prosthesis 96 and augment 70. For example, an augment 70 having a 48 mm inner diameter can be utilized in conjunction with acetabular prostheses 96 that respectively have an outer diameter of 40, 50, and 52 mm.

The outer diameter of augment 70 is generally configured to correspond to a reamed diameter of bone so that acetabular augment 70 can be press-fit within the bone, as illustrated in FIG. 16B. In this regard, reamer head 42 can be provided in multiple different sizes to correspond with the acetabular augment 70 outer diameter. However, in order to ensure the reamer head 42 reams an appropriate distance from a center of acetabulum, reamer assembly 40 is adjustable between a plurality of different positions each corresponding to a particular size acetabular cup prosthesis 96 and a particular size acetabular augment 70, as described further below. The size of an acetabular cup prosthesis 96 is nominally associated with its diameter, and the size of an acetabular augment 70 is nominally associated with its thickness 76.

In this regard, an exemplary kit may include a plurality of acetabular cup prostheses 96 ranging from a size 48 to a size 72 in two-millimeter increments. The kit may also include acetabular augment 70 groupings of various sizes based the outer diameter of each group. A plurality of such groupings may be provided in the kit such that each grouping has three different sizes of augment 70 with the same outer diameter. For example, a first group may have a size 15, 20, and 25 augment each with a 46 mm outer diameter. Thus, this first group includes a first augment 70 with a 15 mm thickness a 46 mm outer diameter, a second augment 70 with a 20 mm thickness and a 46 mm outer diameter, and a third augment 70 with a 25 mm thickness and a 46 mm outer diameter. The kit may include additional similar groupings. For example, second, third, fourth, fifth, and sixth groupings may each include size 15, 20, and 25 augments with outer diameters of 50, 54, 58, 62, and 66 mm, respectively.

The kit may also include a plurality of scoops 22, reamer heads 42, and tool guides 50 to accommodate the multitude of possible acetabular and augment size combinations. Thus, the scoops 22 of the kit may each have a diameter corresponding to an acetabular cup 96 within the kit, and the reamer heads 42 may each have a diameter corresponding to an outer diameter of the augments 70 in the kit. Additionally, a plurality of tool guides 50 may be provided each associated with a different size augment 70. For example, a first tool guide 50 may be associated with a size 15 augment 70, a second tool guide 50 may be associated with a size 20 augment 70, and a third tool guide 50 may be associated with a size 25 augment 70. This allows acetabular wedge augment reaming tool 10 to be highly adjustable while minimizing the number of instruments and bulk of the instruments in the operating theater.

Acetabular wedge augment reaming tool 10 is adjustable to help accommodate the various size augments 70 in the kit. In this regard, each tool guide 50 includes sizing markings 60a-b located on the proximal surface 54a of the guide 54 and the proximal surface 51 of the housing 51, as best shown in FIGS. 9A and 9B. The markings 60a-b correspond to a position of the reamer assembly 40 along guide axis GA and also correspond to various sizes of acetabular cup prostheses and various acetabular augment outer diameters.

For example, in the embodiment depicted in FIG. 9A, tool guide 50 is associated with a size 15 augment. A first set of exemplary augment and acetabular cup markings 60a are shown in FIGS. 9A and 9B. The markings 60a include first, second, and third acetabular augment markings 62, 64, 66 and associated with different acetabular augment outer diameters within the size 15 augments. In this regard, a first marking 62 is associated with a 46 mm outer diameter size 15 augment 70, a second marking 64 is associated with a 50 mm outer diameter size 15 augment 70, and a third marking is associated with a 54 mm outer diameter size 15 augment 70.

Additionally, multiple groupings of acetabular cup size markings 62a-c, 64a-d, and 66a-d are located on the second sidewall 51b of housing 51 adjacent to augment markings 62, 64, and 66. More specifically, a first group of acetabular cup size markings 62a-c are respectively associated with 48, 50, and 52 mm acetabular cup prostheses, a second group of markings 64a-d are respectively associated with 50, 52, 54, and 56 mm acetabular cup prostheses, and a third group of acetabular cup size markings 66a-d are respectively associated with 54, 56, 58, and 60 mm acetabular cup protheses. Thus, as an example, when an operator selects a size 15 acetabular augment with an outer diameter of 54 mm to be implanted with an acetabular cup prosthesis of 56 mm, the operator rotates knob 57a until marking 66, which is associated with the 54 mm augment, is aligned with marking 66b which is associated with the 56 mm acetabular cup prosthesis, as shown in FIG. 9.

Tool guide 50 also includes a second set of acetabular augment and cup markings 60b at an opposite side of tool guide 50 which are similar to the first set of markings 60a but corresponding to different size augments and acetabular cup prostheses. Thus, system 10 has the ability to adjust to a multitude of acetabular cup and augment combinations. This is facilitated at least by the parallel movement of reamer assembly 40 relative to scoop assembly 20.

In addition to that described above and illustrated in the figures, various other operations will now be described. It should be understood that the following operations do not have to be performed in the exact order described below. Instead, various steps may be handled in a different order or simultaneously. Steps may also be omitted or added unless otherwise stated therein.

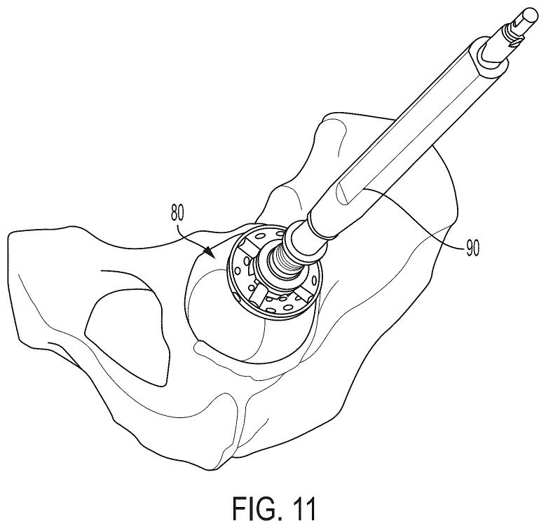

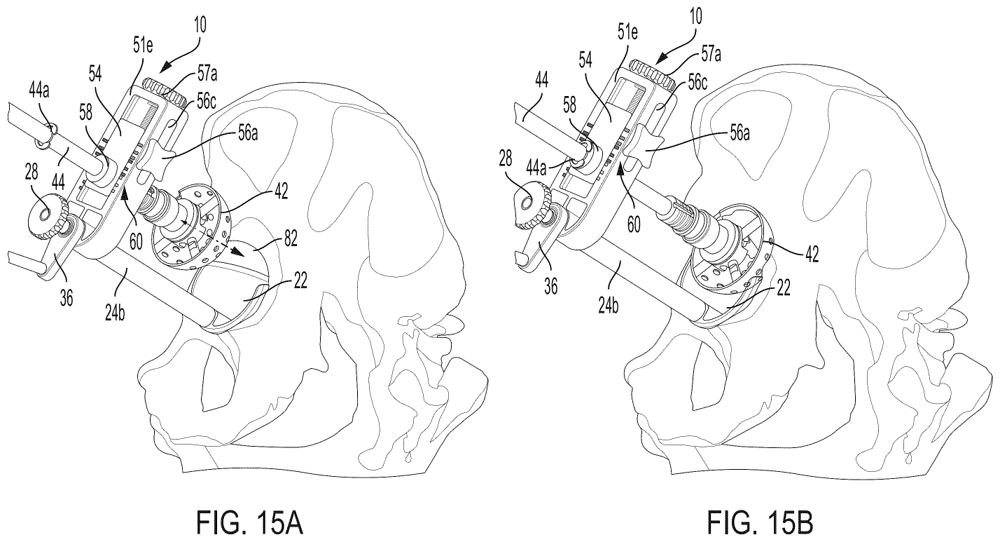

FIGS. 11-16B illustrate a method of using the acetabular wedge augment reaming tool 10. Such method may be conducted to augment an acetabular cup prosthesis involving a cavitary defect, such as in a revision procedure.

As shown in FIG. 11 an axial reamer 90 is used to ream an acetabulum 80.

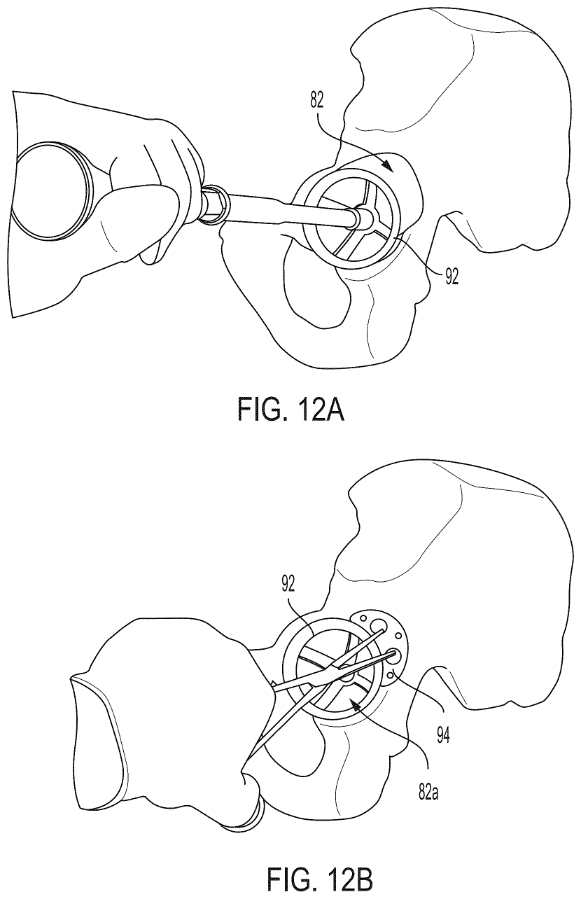

Thereafter, trialing the reamed acetabulum 80 using an acetabular shell trial 92 to determine a first reamed acetabulum diameter is performed, as shown in FIG. 12A. An augment trial 94 is then placed adjacent to acetabular shell trial 92 and within superior bone defect 82 to determine an appropriate size and outer diameter of a final augment prosthesis, as shown in FIG. 12B. In this regard, it may be determined that a desired augment has a first size and a first outer diameter.

Once trialing is performed, a scoop 22 with a diameter equivalent to that of the reamed acetabular diameter determined during the trialing step is selected. The selected scoop 22 is then assembled to the scoop assembly 20 by mating the scoop modular connection 26c with the scoop shaft modular connection 26a-b. In this regard, the hex feature 26b of scoop outer shaft 24b is placed into modular connection 26c of scoop 22, and then threaded end 26a of inner shaft 24a is inserted through scoop outer shaft 24b and into engagement with modular connection 26c. Thereafter, scoop knob 22 is rotated to threadedly connect inner scoop shaft 24a and scoop 22 together. The hex connection between scoop 22 and outer scoop shaft 24b prevents rotation of scoop 22 relative to inner and outer shafts 24a-b as inner shaft 24a is rotated.

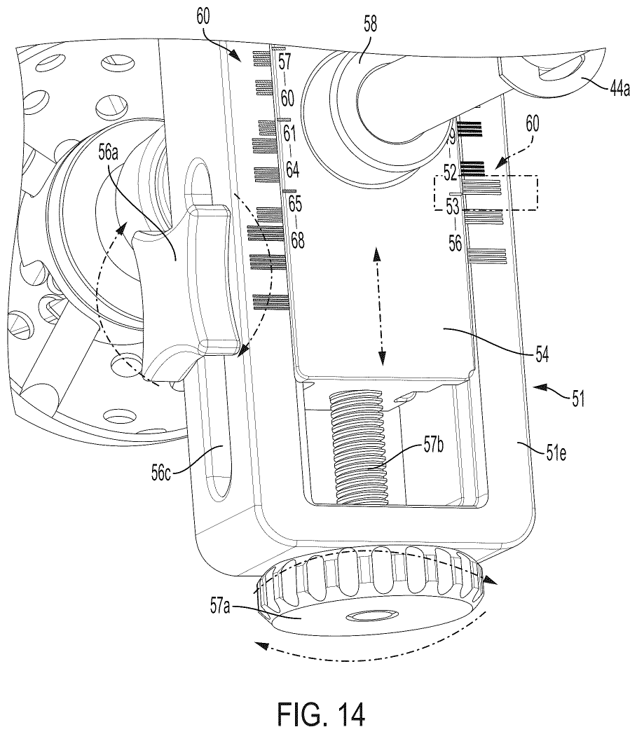

Reamer head 42 may be connected to distal end of reamer shaft either before or after connecting scoop 22 to scoop shafts 24a-b. Reamer head 42 is selected to have a diameter corresponding to the first diameter of the augment 70 determined from trialing the defect 82. It should be noted that the tool guide 50 is selected based on the augment size. Tool guide 50 may have a marking on it to indicate the size, such as size 15, 20, or 25, for example. Reamer guide 54 is then adjusted for the outer diameter of the augment 70 as determined in the augment trialing step. FIG. 14 depicts the adjustment of the reamer guide 54 using the adjustment knob 57a. In this regard, adjustment knob 57a may be rotated in a clockwise direction to position the reamer guide 54 in a vertically upward direction within the housing 51, and the adjustment knob 70a may be rotated in a counterclockwise direction to position the reamer guide 54 in a vertically downward direction within the housing 51. Once the reamer guide 54 is in the desired position as indicated by the relative positioning of the acetabular and augment size markings 60, as described in detail above, the locking knob 56a may be rotated to lock the reamer guide 54 in place within the housing 51. Such locking will prevent any unwanted movement or vibration of the reamer guide 54 during an operation.

Scoop 22 is then inserted into the first reamed acetabulum 80a which sets the position of reamer assembly 40. Because the scoop outer diameter matches that of the reamed acetabulum, scoop 22 helps hold system 10 in place. However, the operator can gain additional positive control by gripping offset handle 30. The tool connector 46 of the acetabulum reamer assembly 40 is connected to a power tool (not shown). The acetabulum reamer assembly 40 is then driven along the reamer axis A3. The acetabulum reamer assembly 40 plunges the acetabular reamer head 42 forward to ream the superior bone defect 82.

FIG. 16A depicts the prepared bone with the reamed acetabulum 84 and reamed offset void 82. As shown, the offset void 82 is entirely concavely curved to correspond to a convexly curved surface 72 of augment 70.

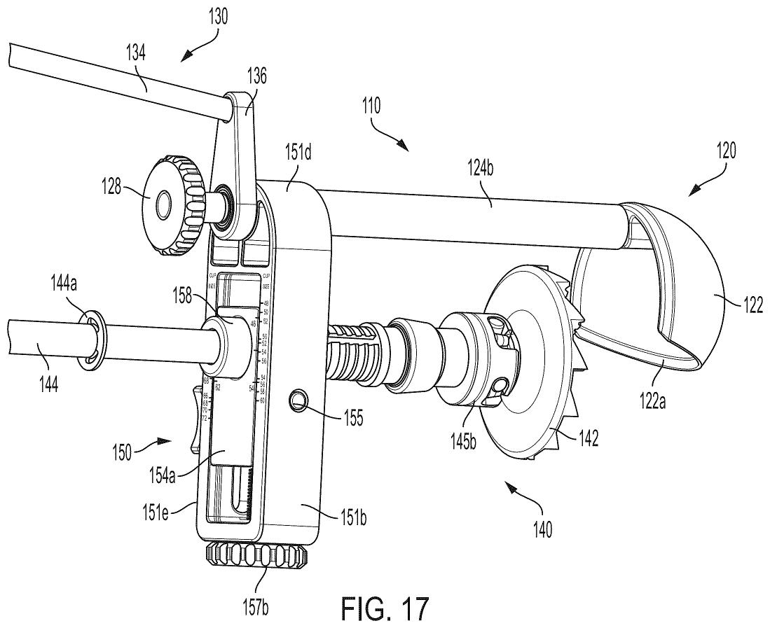

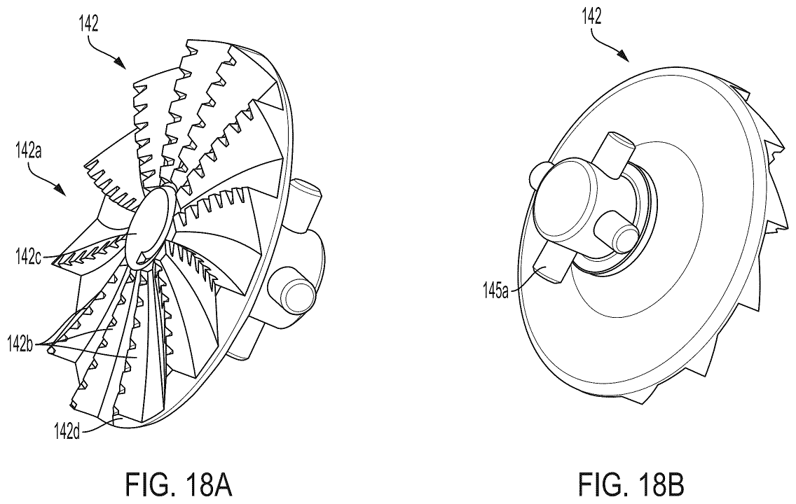

FIGS. 17-18B depict an acetabular wedge augment reaming tool 110 according to another embodiment of the present disclosure. For ease of review, like elements will be accorded like reference numerals to that of acetabular wedge augment reaming tool 10 but within the 100-series of numbers. Acetabular wedge augment reaming tool 110 similarly includes a scoop assembly 120, a handle assembly 130, a tool guide 150, and an acetabular reamer assembly 140. Additionally, reamer assembly 140 includes a reamer head 142 and an acetabular reamer shaft 144 modularly connected to reamer head 142 via an acetabular reamer tool connector 145a-b. However, the acetabular wedge augment reaming tool 110 differs with respect to reaming head or cutting head 142. Acetabular reamer head 142 has a planar reaming surface 142a with an array of circumferential cutting features 142b. These cutting features 142b extend from a center 142c to an edge 142d of the planar reaming surface.

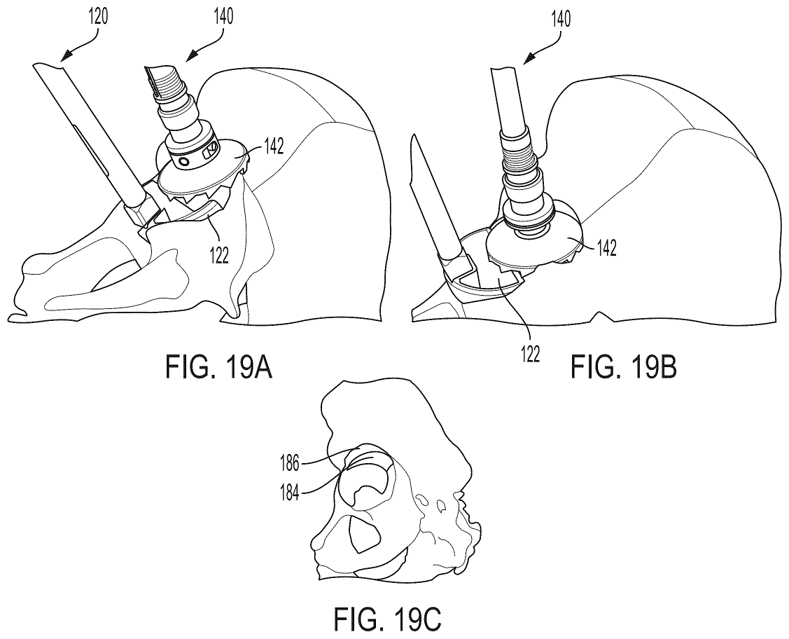

Acetabular wedge augment reaming system 110 is particularly configured for addressing segmental defects of an acetabulum. In this regard, when reamer head 142 is driven into bone adjacent an acetabulum, as shown in FIGS. 19A and 19B, reamer head 142 forms a planar bottom surface 184 with a concave sidewall 186, as best shown in FIG. 19C. Such planar and concave surfaces 184, 186 correspond to a planar and convex surface 71, 72 of augment 70.

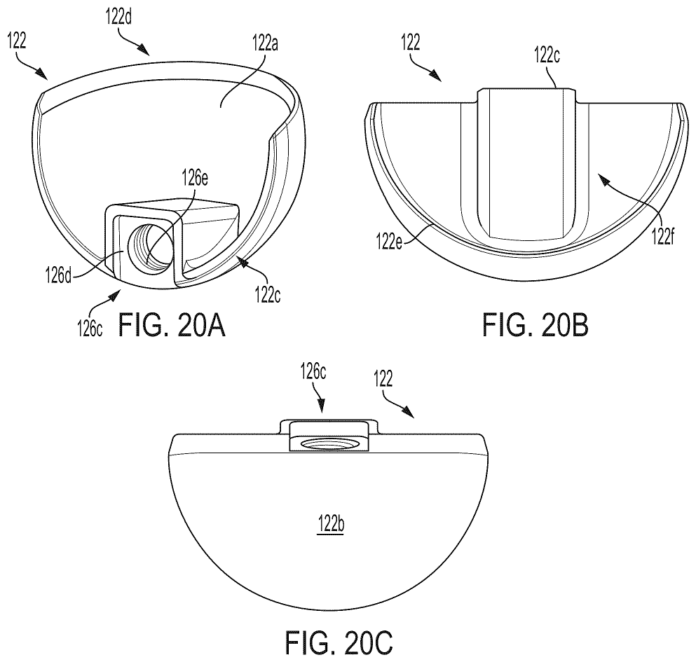

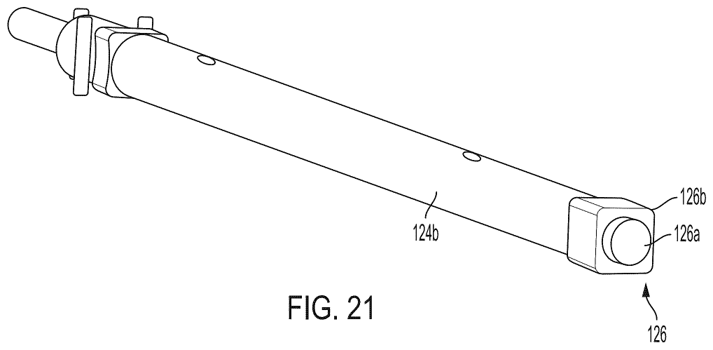

FIGS. 20A-20C depict an alternative scoop or indexer head 122. Scoop 122 is like scoop 22 except with respect to modular connection 126c which may be in the form of a square cavity 126d with a threaded opening 126e. Additionally, FIG. 21 depicts an alternative outer scoop shaft 124b configured to a engage the square cavity 126d of scoop 122. In this regard, outer scoop shaft 124b has a square shaped anti-rotation feature 126b at its distal end that matches the square-shaped cavity 126d in scoop 122. Similar to scoop assembly 20, a threaded tip 126a of an inner scoop shaft extends through the distal end of outer scoop shaft 124b to engage a threaded opening 126e in scoop 122. Such assembly helps provide stability to scoop 122 during operations, as described above with respect to scoop assembly 20.

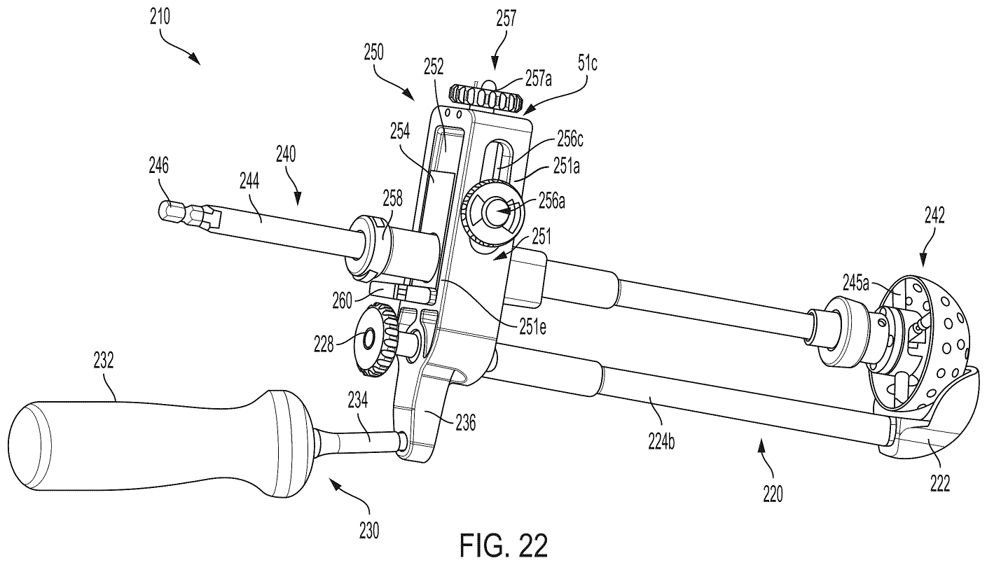

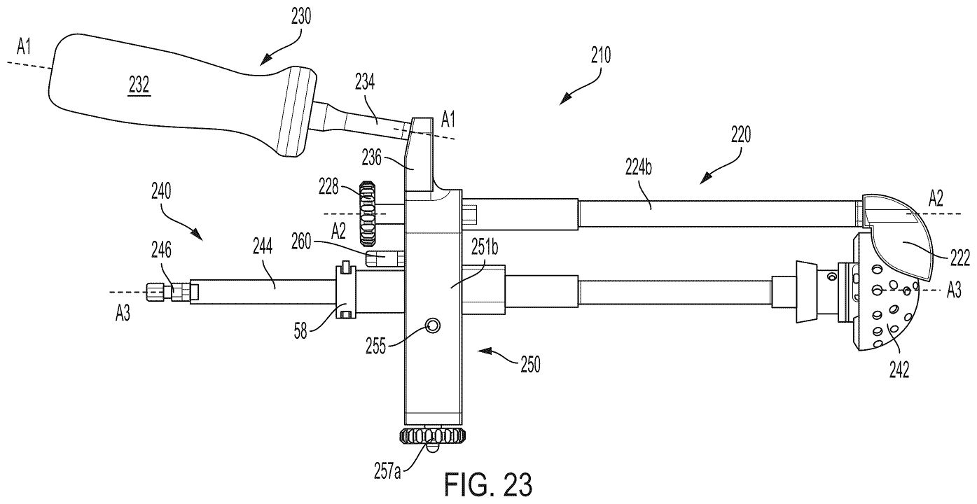

FIGS. 22-25C depict an acetabular wedge augment reaming tool 210 according to another embodiment of the present disclosure. For ease of review, like elements will be accorded like reference numerals to that of acetabular wedge augment reaming tools 10, 110 but within the 200-series of numbers. Acetabular wedge augment reaming tool 210, similar to tools 10, 110, includes a scoop assembly 220, a handle assembly 230, a tool guide 250, and an acetabular reamer assembly 240. Additionally, acetabular wedge augment reaming tool 210 includes a shim 260 configured to be disposed within a cavity 252 of a housing 251 of tool guide 250. In this embodiment, shim 260 is used as an alternative to or in combination with sizing markings 60a-b as included in acetabular wedge augment reaming tool 10. Further, as previously described for acetabular wedge augment reaming tools 10, 110, acetabular wedge augment reaming tool 210 is configured for use in addressing cavitary defects of an acetabulum. A bone surface prepared using tool 210 may receive the acetabular augment 70, an example of which is depicted in FIG. 10, and the acetabular cup prosthesis 96, an example of which is depicted in FIG. 16B.

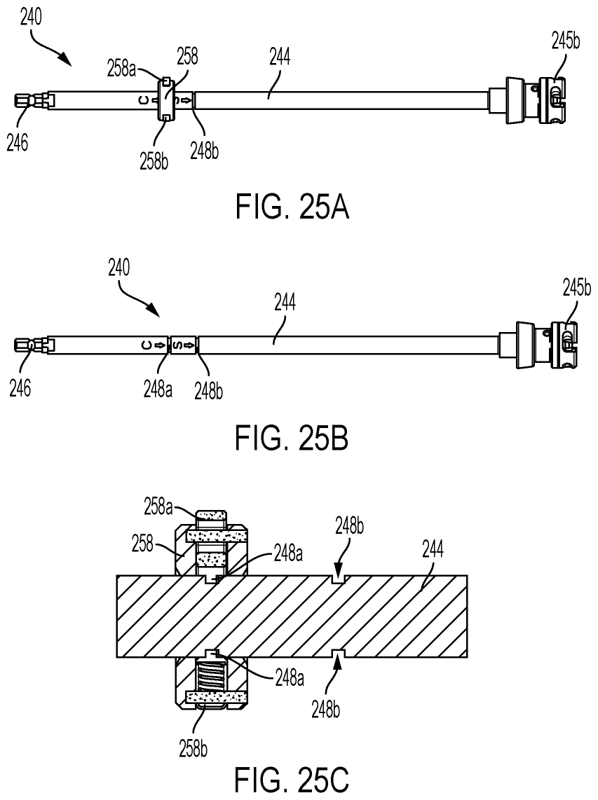

In FIGS. 25A-25C, acetabular reamer assembly 240 of acetabular wedge augment reaming tool 210 is shown in insolation. Acetabular reamer assembly 240 includes a shaft 244 with a first groove 248a and a second groove 248b, each extending around a circumferential surface of the shaft and being spaced apart along a length of the shaft. Each groove 248a, 248b is configured to be selectively and independently engaged by mechanical stop 258 to prevent the unrestricted sliding movement of the acetabular reamer assembly 240 within the opening of the reamer guide 254 of the tool guide 250 when the acetabular reamer assembly is disposed in the reamer guide. In other words, mechanical stop 258 acts as a depth limiter when positioned in either groove 248a, 248b in that axial advancement of the acetabular reamer assembly 240 through the opening of the reamer guide 254 is limited by contact between mechanical stop 258 and a proximal surface of reamer guide 254. Mechanical stop 258 includes engagement features 258a, 258b configured to allow a user to releasably engage either groove 248a, 248b with mechanical stop. In one example, the engagement features 258a, 258b may include one or more springs biasing a portion of the mechanical stop 258 in the selected groove. Compression of the engagement features 258a, 258b and one or more springs may release the fixation of the mechanical stop 250 to the selected groove and may allow mechanical stop 258 to be slidably movable along shaft 244.

Each groove 248, 248b may correspond to a shaft length compatible with a specific reamer head type to be used with acetabular wedge augment reaming tool 210. For example, the mechanical stop 258 may be positioned within the first groove 248a to use a conical acetabular reamer head 142, as best shown in FIG. 17. Alternatively, the mechanical stop 258 may be positioned within the second groove 248b to use a spherical acetabular reamer head 242, as best shown in FIG. 22. While two grooves are shown on shaft 244 in the depicted embodiment, any number of grooves may be included on the shaft to provide options for limiting the advancement depth of the of acetabular reamer assembly within the reamer guide. In this manner, the acetabular reamer assembly 240 may be adapted for use with any number and type of acetabular head.

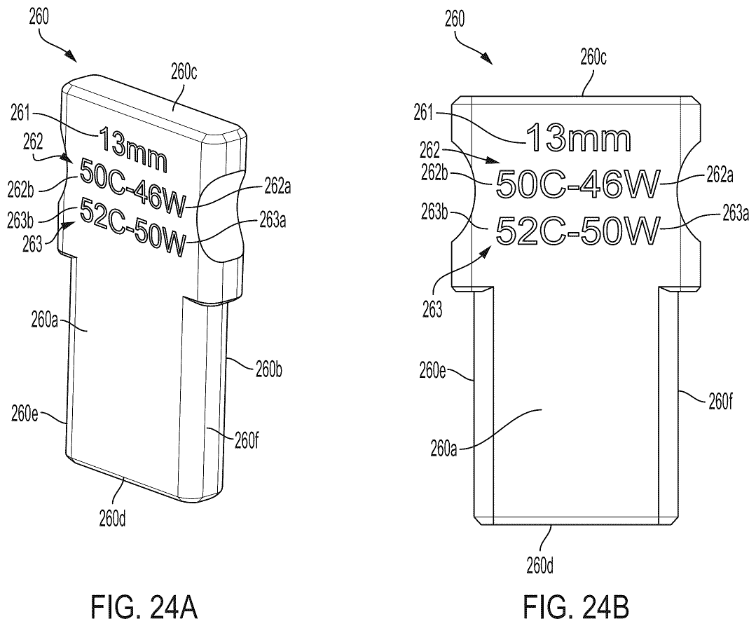

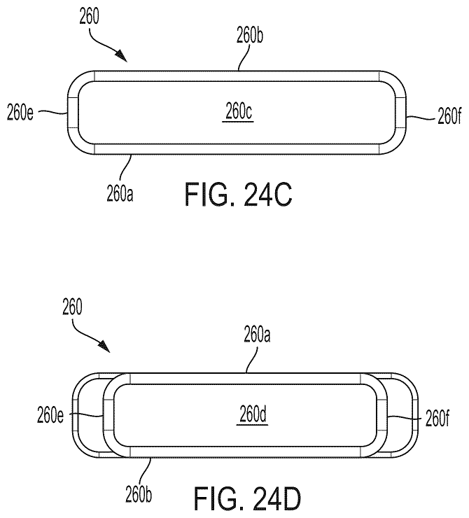

Shim 260 is substantially rectangular in shape and includes a front or top surface 260a opposite a bottom or rear surface 260b. Front and rear surface 260a, 260b are separated by a substantially planar proximal surface 260c and a substantially planar distal surface 260d. Further, shim 260 includes first and second sides 260e, 260f, each of which include a concave portion closer to the proximal surface 260c to assist a user for gripping the shim, as discussed further below. In variations, a shape of an outer surface of the shim may vary from that of FIGS. 24A-24D.

Within acetabular wedge augment reaming tool 210, shim 260 is positionable directly between a wall of cavity 252 and a reamer guide 254 slidably disposed within cavity 252. In position within the cavity, shim 260 sets a position of acetabular reamer assembly 240 relative to scoop assembly 220. This in turn allows for the use of an acetabular augment 70 and acetabular cup 96 with respective sizes complementary to the shim. Acetabular wedge augment reaming tool 210 is configured for use with shims of different sizes so that a shim may be chosen to provide a desirable spacing between scoop assembly and acetabular reamer assembly.

As shown in FIGS. 24A-B, shim 260 includes markings 261, 262, 263 on the first or top surface 260a of shim. In the embodiment depicted in FIGS. 24A-24B, shim 260, as used with tool 210, is associated with an augment having a 13 mm thickness or width, or size 13 augment, as indicated by augment thickness marking 261. In one example based on augment 70 shown in FIG. 10, a thickness or width of the augment is thickness 76. Markings 262, 263 are associated with different groupings of an acetabular augment 70 and an acetabular cup prothesis 96. For example, marking 262 is associated with a first acetabular augment outer diameter and a first acetabular cup prothesis diameter, such combination being further associated with the augment thickness. Similarly, marking 263 is associated with a second acetabular augment outer diameter and a second acetabular cup prothesis diameter, such combination being further associated with the augment thickness. However, shim 260 is not limited to only markings 262, 263 and may include additional markings for association with additional augments and additional cups. This may be applicable for the same shim or for shims having different sizes. In one example based on augment 70 shown in FIG. 10, an augment outer diameter is defined by a convex outer surface 72 of the augment 70. Further, in one example based on acetabular cup prosthesis 96 shown in FIG. 16B, an acetabular cup prosthesis diameter is associated with an outer diameter of the acetabular prosthesis 96.

Marking 262 includes an augment marking 262a and an acetabular cup marking 262b. Marking 262a is associated with an acetabular augment outer diameter and marking 262b is associated with an acetabular cup prothesis diameter. Use of shim 260 with acetabular wedge augment reaming tool 210 to prepare the bone in the hip allows for the implantation of a first acetabular augment having an outer diameter indicated by marking 262a in combination with a first acetabular cup prothesis having a diameter or size indicated by marking 262b. In this regard, for shim 260 as depicted, marking 262a indicates that shim 260 is associated with an augment 70 having a 46 mm outer diameter and marking 262b indicates that shim 260 is associated with an acetabular cup prosthesis 96 having 50 mm outer diameter. Further, marking 263 similarly includes an augment marking 263a and an acetabular cup marking 263b for a second acetabular augment and second acetabular cup prothesis, respectively. In this regard, for shim 260 as depicted, marking 263a indicates that shim 260 is associated with an augment 70 having a 50 mm outer diameter and marking 262b indicates that shim 260 is associated with an acetabular cup prosthesis 96 having 52 mm diameter. In this way, the first set of markings 262a-b is for an augment having a thickness of 13 mm and a 46 mm outer diameter and an acetabular cup having a 50 mm diameter, and the second set of markings 263a-b is for an augment having a thickness of 13 mm and a 50 mm outer diameter and an acetabular cup having a 52 mm diameter. Thus, as an example, when an operator selects an acetabular augment with a thickness of 13 mm and an outer diameter of 46 mm to be implanted in conjunction with an acetabular cup prosthesis with a 50 mm diameter, the operator may dispose shim 260 within the cavity 252 of tool guide 250 and adjust the reamer guide 254 using the adjustment knob 257a until the reamer guide 254 is in the desired position abutting the shim 260. Once the reamer guide 254 is in the desired position against the shim 260, the locking knob 256a may be rotated to lock the reamer guide 254 in place within the housing 251, thereby fixing the reamer guide 254 and shim 260 in place within tool guide 250.

In some embodiments, a kit may include one or more components from acetabular wedge augment reaming tool 210. For example, the kit as previously described for acetabular wedge augment reaming tool 10 may further include a plurality of shims 260. Thus, such kit may include a plurality of shims 260, acetabular cups 96, acetabular augments 70, scoops 222, reamer heads 242, and a tool guide 250. In this example, the plurality of shims may include shims having different thicknesses to produce different separations between the reamer shaft and the scoop shaft when positioned in a cavity of the tool guide. Put another way, when a kit includes two or more shims 260 having different sizes, a user may select a shim with a size that best complements a desired augment and acetabular cup size determined to be most suitable as an implant. Thus, a plurality of shims 260 may be included as part of a kit, each shim being associated with at least one complementary augment and acetabular cup combination. In further examples, a kit may include a tool guide 250 and at least a set of two of one or more of reamer head 242, scoop 222, acetabular augment 70, acetabular cup 96 and shim 260. In yet another example, a kit may include at least a set of two of one or more of reamer head 242, scoop 222, acetabular augment 70, acetabular cup 96 and shim 260. The aforementioned kits may also include one or both of an acetabular reamer assembly 240 and a scoop assembly 220. In any one of the contemplated kits, sets of reamer heads 242, scoops 222, acetabular augments 70, acetabular cups 96 and shims 96 may include within such sets applicable components having different sizes. In any one of the above kits that include a tool guide 250, such kit may include one or more reamer guides 254. Where two or more reamer guides 254 are included, such reamer guides may have different sizes including different sized openings to receive a reamer assembly 240.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acetabular wedge augment reaming tool, comprising:
a scoop assembly having a scoop configured to be received within an acetabulum and a scoop shaft extending from the scoop;
an acetabular reamer assembly having a reamer head and a reamer shaft extending from the reamer head; and
a tool guide comprising a reamer guide housing and a reamer guide positioned within the reamer guide housing and moveable along a first axis relative to the reamer guide housing,
wherein, the reamer shaft of the acetabular reamer assembly is slidably connected to the reamer guide such that it is moveable along a second axis relative to the reamer guide and the scoop shaft of the scoop assembly is connected to the reamer guide housing,
wherein, moving the reamer guide along the first axis moves the reamer assembly closer to or further away from the scoop assembly.

2. The acetabular wedge augment reaming tool of claim 1, wherein the scoop is modularly connected to a distal end of the scoop shaft of the scoop assembly and the scoop assembly includes a housing shaft connected to and extending from the reamer guide housing, the scoop shaft being received within the housing shaft so that the distal end of the scoop shaft extends from a distal end of the housing shaft.

3. The acetabular wedge augment reaming tool of claim 1, wherein the scoop further includes a semi-spherical exterior that defines an outer diameter and a semi-spherical cavity that defines an inner diameter.

4. The acetabular wedge augment reaming tool of claim 1, wherein the scoop includes a rim extending outwardly from a perimeter of the scoop that is interrupted by a cutout and the scoop shaft is modularly connected to a first side of the scoop and the cutout is positioned at a second side of the scoop opposite the first side.

5. The acetabular wedge augment reaming tool of claim 2, wherein a scoop knob is disposed at a proximal end of the scoop shaft, and the distal end of the scoop shaft is threaded for threaded connection with the scoop.

6. The acetabular wedge augment reaming tool of claim 1, further comprising a handle assembly having a handle and a handle shaft connected to the reamer guide housing, wherein handle assembly extends proximally from the reamer guide housing at an angled offset.

7. The acetabular wedge augment reaming tool of claim 1, wherein the acetabular reamer shaft includes a mechanical stop positioned along a length thereof and configured to limit sliding movement thereof relative to the reamer guide.

8. The acetabular wedge augment reaming tool of claim 1, wherein the reamer housing includes housing rails at least partially defining a cavity within the reamer housing, the reamer guide being positioned within the cavity and engaging the housing rails from within the cavity of the reamer housing.

9. The acetabular wedge augment reaming tool of claim 8, further comprising an adjustment knob and a threaded shaft extending from the adjustment knob into the cavity of the housing and engaging the reamer guide such that rotation of the adjustment knob slides the reamer guide along the housing rails and along the first axis.

10. The acetabular wedge augment reaming tool of claim 1, wherein the scoop has a convex exterior having a first diameter corresponding to a reamed diameter of an acetabulum.

11. The acetabular wedge augment reaming tool of claim 10, wherein the reamer head has a second diameter equal to the first diameter.

12. The acetabular wedge augment reaming tool of claim 1, wherein a reamer guide knob is configured on a first side of the reamer guide housing and locks the reamer guide in a desired reaming position.

13. The acetabular wedge augment reaming tool of claim 1, wherein a locking pin is positioned on a second side of the reamer guide housing and is moveable from a first position in which the reamer guide is moveable relative to the reamer guide housing and a second position in which the locking pin locks the reamer guide in a desired reaming position.

14. The acetabular wedge augment reaming tool of claim 1, wherein the reamer head is spherical or planar.

15. The acetabular wedge augment reaming tool of claim 1, further comprising a shim positioned within a cavity of the reamer guide housing, the shim being configured to limit movement of the reamer guide along the first axis.

16. The acetabular wedge augment reaming tool of claim 15, wherein the shim is sized such that when the shim is positioned within the cavity of the reamer guide housing to define a spacing between the scoop assembly and the acetabular reamer assembly, operation of the scoop assembly and the acetabular reamer assembly on a bone surface produces surfaces sized to receive an acetabular wedge augment having a first predetermined size and an acetabular cup having a second predetermined size.

17. A reaming tool, comprising:
a guide assembly having a housing and a reamer guide, the housing having a first end, a second end, and a channel located between the first and second ends, the reamer guide being disposed within the channel and moveable therein along a first axis;
a reaming assembly having a reamer head and a reamer shaft extending from the reamer head, an elongate dimension of the reamer shaft extending along a second axis and being coupled to the reamer guide; and
an indexing assembly having an indexing head configured to be received within a bone void and an indexing shaft extending from the indexing head, an elongate dimension of the indexing shaft extending along a third axis and being coupled to the reamer guide housing,
wherein moving the reamer guide along the first axis from a first position to a second position moves the reamer assembly relative to the indexing assembly, and
wherein the second and third axes are parallel in the first and second positions.

18. The reaming tool of claim 17, wherein the first axis is perpendicular to the second and third axes.

19. A kit for an acetabular wedge augment reaming tool, comprising:
a scoop assembly including a scoop configured to be received within an acetabulum, the scoop having a diameter corresponding to a diameter of an acetabular cup implant;
an acetabular reamer assembly having a reamer head, the reamer head having a diameter corresponding to a diameter of an acetabular augment;
a tool guide comprising a reamer guide housing and a reamer guide configured to be positioned within the reamer guide housing; and
a plurality of shims, each shim being configured to be positioned within a cavity of the reamer guide housing, and each shim being configured to limit movement of the reamer guide within the reamer guide housing,
wherein the scoop assembly and the acetabular reamer assembly are adapted for operative connection to the tool guide along with a first shim of the plurality of shims such that the first shim is disposed between the scoop assembly and the acetabular reamer assembly.

20. The kit of claim 19, wherein the reamer guide is slidable within the reamer guide housing such that a second shim of the plurality of shims having a different size than the first shim is disposable between the scoop assembly and the acetabular reamer assembly.
